(19) United States
(12) Patent Application Publication
(10) Pub. No.: US 2024/0390101 A1
(43) Pub. Date: Nov. 28, 2024

(54) MEDICAL EQUIPMENT MOUNTING SYSTEM

(71) Applicant: SLD Technology, Inc., Portland, OR (US)

(72) Inventors: Kevin Joseph SCHREIBER, Portland, OR (US); Jason WILKINSON, Battleground, WA (US); Matthew YUNG, Sandy, OR (US); Mikah BARNETT, Clackamas, OR (US)

(21) Appl. No.: 18/674,556

(22) Filed: May 24, 2024

Related U.S. Application Data
(60) Provisional application No. 63/504,080, filed on May 24, 2023.

(51) Int. Cl. A61B 90/35; A61G 12/00

(52) U.S. Cl. CPC A61B 90/35; A61G 12/004

(57) ABSTRACT

Systems for mounting medical equipment in a configurable manner are provided. In one example, a mounting system comprises a clamping portion configured to removably couple to a truss structure. The clamping portion has a first mounting support and a second mounting support. The first and second mounting supports are configured to couple to each other on opposite sides of a portion of the truss structure. The second mounting support has features complimentary to a shape of the truss structure to allow sliding engagement of the truss structure. The mounting system further comprises an engagement portion having at least one opening. The one opening is configured to receive one or more fasteners to couple to an equipment mount, and the engagement portion extends from the first mounting support of the clamping portion.

### CROSS-REFERENCE TO RELATED APPLICATIONS

This application claims the benefit of U.S. Provisional Application No. 63/504,080, filed May 24, 2023, the entire disclosure of which is incorporated by reference herein.

### BACKGROUND

Hospital operating rooms typically include surgical equipment and one or more lights that are mounted over a surgical site. The surgical equipment may be movable in relation to a surgical site target zone (e.g., equipment on a movable arm mounted to the ceiling).

In many mounting systems, the mounting arrangement for the surgical equipment is customized with a fixed arrangement for the mounting locations. Accordingly, if new equipment is to be installed or equipment is to be moved to different locations in the operating room, a new mounting arrangement is required, which may result in having to replace portion of, or even the entire existing mounting arrangement. Alternatively, costly changes may have to be made to accommodate the new equipment or the new equipment position.

Moreover, because the surgical equipment (e.g., surgical light) may be positioned directly over the surgical target zone, the surgical equipment may block airflow generated by the air supply arrangement and create a low pressure zone underneath the surgical equipment. The low pressure zone causes air turbulence underneath the surgical equipment. Due to turbulent airflow, various contaminants generated through a surgical procedure may be circulated within the surgical environment. For example, surgical staff may carry particulate and bacterial contaminants that may be dispersed directly above a surgical site in the absence of filtered, downward, unidirectional flow. Further, bone fragments, biological fluids, and blood may be projected upward toward the surgical equipment, which is cleaned and sterilized between surgical procedures.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems are described herein for mounting medical equipment in a configurable manner. In one example, a mounting system comprises a clamping portion configured to removably couple to a truss structure. The clamping portion has a first mounting support and a second mounting support. The first and second mounting supports are configured to couple to each other on opposite sides of a portion of the truss structure. The second mounting support has features complimentary to a shape of the truss structure to allow sliding engagement of the truss structure. The mounting system further comprises an engagement portion having at least one opening. The one opening is configured to receive one or more fasteners to couple to an equipment mount, and the engagement portion extends from the first mounting support of the clamping portion.

In one example, an airframe system comprises a frame defining a supply air array having a plurality of removable portions. The plurality of removable portions are positioned at airflow inlets of the supply air array and covering the airflow inlets. The airframe system further comprises one or more equipment mounts movably mounted to a perimeter of the frame.

### BRIEF DESCRIPTION OF THE DRAWINGS

FIG. 1 illustrates a lateral view of an operating room according to an example.

FIG. 2 illustrates a perspective bottom view of a supply air array according to one or more examples.

FIG. 3 illustrates a perspective bottom view of a mounting arrangement according to various examples.

FIG. 4 illustrates a mounting system with an equipment mount coupled in one position according to an example.

FIG. 5 illustrates a mounting system with an equipment mount coupled in another position according to an example.

FIG. 6 illustrates a mounting system with an equipment mount coupled in another position according to an example.

FIG. 7 is an exploded view of components of a mounting system according to an example.

FIG. 8 illustrates components of a mounting system coupled together according to an example.

FIG. 9 is another exploded view of components of a mounting system according to an example.

FIG. 10 illustrates components of a mounting system coupled together according to an example.

FIGS. 11-14 illustrate a bracket arrangement according to an example.

FIG. 15 illustrates removable portions of a supply air array according to an example.

FIG. 16 illustrates microtabs of the removable portions of FIG. 15.

FIG. 17 illustrates a configuration of equipment mounts with an exposed airflow inlet according to an example.

FIG. 18 illustrates a configuration of equipment mounts with a supply air array according to an example.

FIG. 19 illustrates configuration of removable panels according to an example.

FIG. 20 illustrates an air ductwork connected to an airflow inlet configuration according to an example.

FIG. 21 illustrates an air ductwork connected to an airflow inlet configuration according to another example.

FIGS. 22 and 23 illustrate a blast plate cartridge according to an example.

FIG. 24 illustrates a movable electrical enclosure configuration according to an example.

FIG. 25 illustrates a movable electrical enclosure configuration according to another example.

FIG. 26 illustrates a predefined configuration of modules for a supply air array according to an example.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The methods and systems disclosed herein, for example, may be suitable for use in different mounting applications, such as in different medical or clinical settings. That is, the herein disclosed examples or can be implemented in combination with equipment configurations or assemblies other than for particular applications, such as other than for particular operating room arrangements (e.g., surgical suites).

Various examples provide a mounting system or arrangement for mounting equipment, such as surgical equipment within an operating room. For example, in various embodiments, a flexible truss arrangement is provided that allows for easy adjustable coupling of surgical equipment (and other devices or members) to different locations within the operating room (e.g., to different locations of a supply air array 106), as well as flexibility to couple different components using the mounting system. In some examples, the mounting arrangement allows for easy movement of adjustable mounting brackets to position or reposition mounting locations within the room. That is, the mounting brackets allow adjustability in multiple dimensions to provide different configurations of mounting arrangements. Additionally, one or more examples also allow for providing air supply at different locations of the supply air array 106. As such, one or more examples are adaptable to many different settings, room configurations, equipment configurations, etc.

For example, interchangeable mounting of access panels, light troffers and boom mounts for surgical equipment is facilitated and easily configurable during installation and setup, as well when changes are desired or needed. That is, one or more adjustment mechanisms or features of the flexible truss arrangement allow for flexibility in mounting different equipment at different desired or required locations of a fixed truss structure. As such, one or more examples provide a flexible truss mounting system that allows for the easy configurable design and redesign (or reconfiguration) of the operating room or medical room (during installation or at a later time) without a change in the mounting structure.

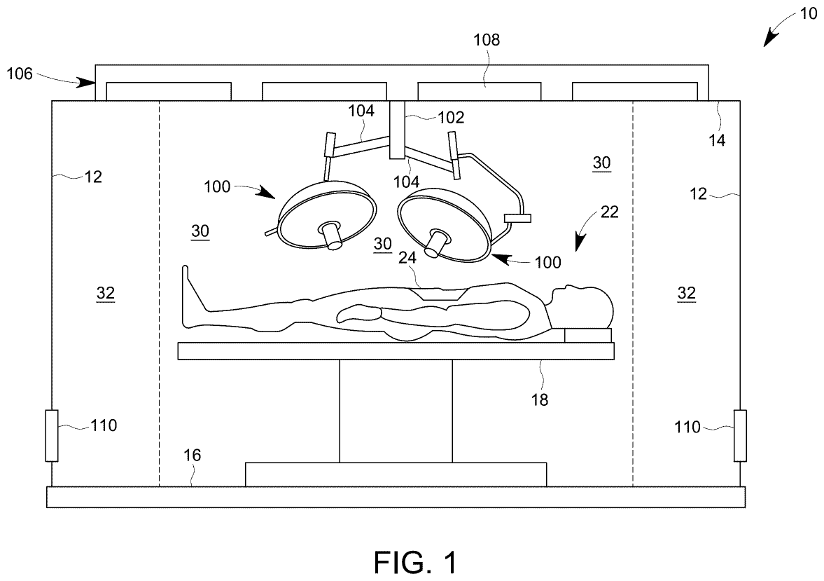

FIG. 1 illustrates a lateral view of an operating room 10, according to one example. The operating room 10 is defined by walls 12, a ceiling 14, and a floor 16. An operating table 18 is supported on the floor 16. The operating table 18 may include a support bed 20 that is configured to support a patient 22. A surgical site 24 may be located on the patient 22.

Surgical equipment, which in the illustrated embodiment is a surgical light system 100 is suspended from the ceiling 14 above the operating table 18, which may define a sterile field 30. A support beam 102 extends downwardly from the ceiling 14 and is coupled to a mounting system and providing a flexible truss system as described in more detail herein. The mounting system in various examples provides an adjustable perimeter truss mounting arrangement that allows for adjustability in different planes and dimensions (e.g., directions, elevations, orientations, etc.) around a perimeter of the sterile field 30. Accordingly, equipment mounts (e.g., mounting plates) are easily positionable at different locations outside of the sterile field 30. In the illustrated example, this adjustability allows for changing the positon of the equipment mount(s) that support one or more boom arms 104 that extend from the support beam 102 into the sterile field 30 (or other equipment). As shown in FIG. 1, two surgical light assemblies 100 may be coupled to two separate and distinct boom arms 104 mounted to different locations on the mounting system, which may be performed in a flexible and configurable manner during installation or thereafter during a reconfiguration or installation of different and/or additional equipment. Thus, more or less surgical light assemblies 100 or other medical equipment than shown may be used and mounted according to one or more examples described herein. The surgical light system 100 is shown only for illustrative purpose and different or additional surgical equipment may be mounted to and suspended from the ceiling 14.

A supply air array 106 (also referred to as an air frame system) is secured to the ceiling 14 and configurable with the mounting system as described in more detail herein. The supply air array 106 may be provided as described, for examples, in U.S. Pat. Nos. 9,903,115, 10,405,952, and 11,259,893. By positioning the mounting plates 42 outside of (along the perimeter) of the supply air array 106, laminar airflow is created directly to the surgical target zone without turbulence that could otherwise be caused by having the mounting plates 42 within the supply air array 106 or within the sterile field 30. The position of the mounting plates 42 are also easily adjustable to further facilitate a frame structure that allows for proper airflow within the surgical target zone even when surgical equipment is positioned above the surgical target zone. For example, in various examples, low pressure zones that could cause air turbulence underneath the surgical equipment are reduced or eliminated. As such, in various examples, because the turbulent airflow is reduced or eliminated, various contaminants generated through a surgical procedure are not circulated within the surgical environment.

One or more examples provide an airframe structure that is configured to channel air, which may be filtered, sterilized or purified, to the surgical target zone with minimal or no air turbulence underneath the surgical equipment and that is mounted as described in more detail herein. Various examples thereby provide an integrated and modular arrangement to effectively deliver airflow directly to the surgical target zone while allowing adjustability in the mounting of equipment along the perimeter of the airframe structure.

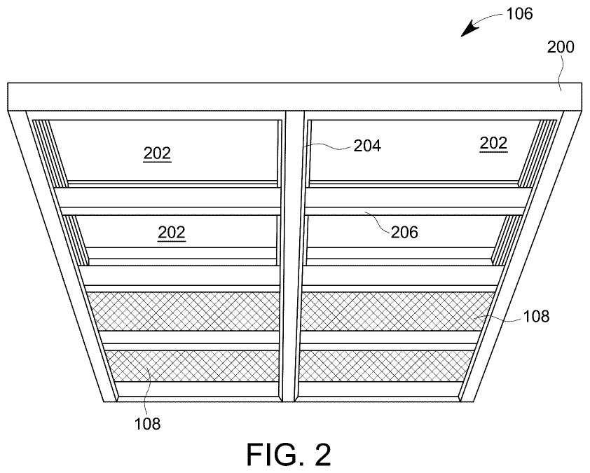

FIG. 2 illustrates a perspective bottom view of the supply air array 106, according to one example. The supply air array 106 in the illustrated example includes a frame 200 (a portion of which is shown and that forms part of a truss structure in various examples) having a plurality of openings 202 defined therein by cross-members 204 and 206. It should be noted that although the illustrated example shows a 2 cell×4 cell array, the supply air array 106 may be sized differently (and pre-defined as described in more detail herein), including having a single opening 202. Additionally, the openings 202 may be sized and shaped differently than illustrated, for example, based on design requirements or constraints. In the example shown in FIG. 2, the front four openings 202 are illustrated with nothing therein and the back four opening 202 illustrate air diffusers 108 coupled within the openings 202. As can be seen, the air diffusers 108 are coupled with the openings 202 such that the air diffusers 108 are recessed within the openings 202 in this example. As described in more detail herein, the frame 200 in various examples defines the supply air array 106 and has a plurality of removable portions, wherein the plurality of removable portions are positioned at airflow inlets of the supply air array and covering the airflow inlets. The frame 200 is also an example or a structure to which the mounting system described herein can be used to provide an adjustable perimeter truss mounting arrangement that allows for adjustability in mounting equipment at many different locations that are easily reconfigurable on the fly.

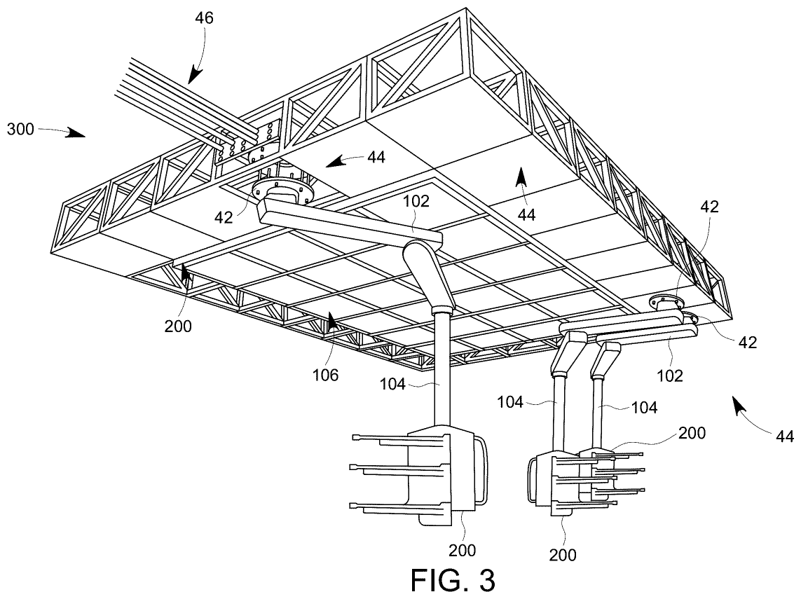

FIG. 3 illustrates a mounting system 300 and shows the mounting plates 42 mounted to different mounting locations 44 defined within the truss structure of the mounting system 300. The mounting system 300 allows for interchangeable mounting of the mounting plates 42 to any location along the perimeter of the frame 200 to thereby define plural mounting locations 44 that are easily adjustable with the mounting system 300 as described in more detail herein. The frame 200 defines the supply air array 106 that can have the adjustable mounting locations 44 and optionally or alternatively have a plurality of removable portions. For example, as described in more detail herein, the plurality of removable portions are positioned at airflow inlets of the supply air array 106 to allow and cover the airflow inlets as described in more detail herein. This configuration provides an adjustable air supply path to the supply air array 106.

Thus, the mounting locations 44 are movably provided along a perimeter of the supply air array 106 and are easily configurable and reconfigurable. Accordingly, for example, the mounting system 300 allows for easy adjustment of the mounting locations 44, as well as the air supply locations. It should be noted that the different mounting locations allow for mounting the mounting plates 42 or, for example, other equipment mounts, thereon. However, the mounting locations 44 also provide interchangeable mounting of different components thereto, such as access panels or light troffers, among other components. For example, the mounting system 300 may be provided on one, two, three, or all four sides of the supply air array 106. As can be seen, the mounting locations 44 may be configured differently, such as to define different mounting types, such as different configurations of mounting plates 42 that include different mounting patterns of bore locations for securing thereto different components (e.g., bolt connection).

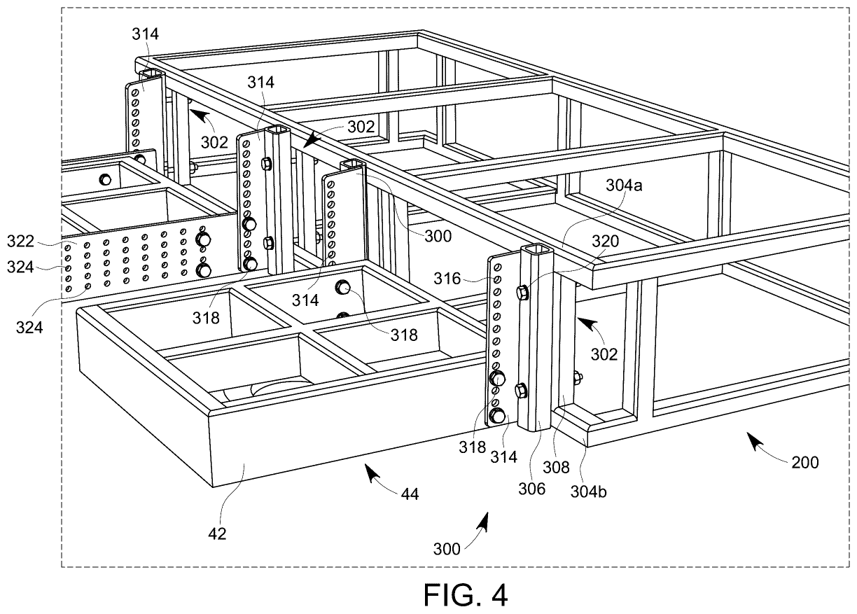

FIGS. 4-6 illustrate the mounting system 300 used to couple mounting plates 42 (or other equipment mounts) to the frame 200 to thereby define multiple mounting locations 44. That is, the mounting system 300 allows for variable positioning of the mounting plates 42 or other equipment mounts along the outside or the perimeter of the frame 200. In some examples, the frame 200 forms part of the supply air array 106 as described in more detail herein. It should be noted that although two mounting plates 42 are shown coupled to the frame 200, additional or fewer mounting plates 42 can be coupled to the frame 200. Additionally, different types of mounting plates 42 can be coupled to the frame 200, as well as other types of equipment mounts, such as for a patient chair lift or support for other medical devices or equipment.

In various examples, the mounting system 300 includes a clamping portion 302 configured to removably couple to a truss structure, such as the frame 200. That is, the clamping portion 302 allows for coupling to the frame 200 at a desired or required location, such as by moving (e.g., sliding engagement of) the clamping portion 302 along upper and lower frame portions 304 of the frame 200. For example, the clamping portion 302 is configured to engage around at least a portion of each of the upper and lower frame portions 304 to secure and support the mounting plate 42 thereto. As can be seen, the clamping portion 302 engages different surfaces of the upper and lower frame portions 304 of the frame 200. As should be appreciated, the clamping portions 302 can also be spaced apart at different distances to allow for coupling of different sized and/shaped mounting plates 42 to the frame 200.

In one or more examples, the clamping portions 302 include a first mounting support 306 and a second mounting support 308. The first and second mounting supports 306, 308 are configured to engage and abut the upper and lower frame portions 304 of the frame 200. That is, the first and second mounting supports 306, 308 are sized and shaped in various examples to be secured to the first mounting support 306 and a second mounting support 308, which is illustrated as an opposing arrangement secured against at least opposite sides of the upper and lower frame portions 304 of the frame 200. As such, the first and second mounting supports 306, 308 are configured to couple to each other on opposite sides of a portion of the truss structure, namely on opposite sides of the upper and lower frame portions 304 of the frame 200.

The engagement of the first and second mounting supports 306, 308 to the upper and lower frame portions 304 of the frame 200 is held in position in part by the configurations of the first and second mounting supports 306, 308. In one example, the second mounting support 308 has ends 310 that are complimentary to a shape of the truss structure to allow sliding engagement to the truss structure. For example, the ends 310 of the first and second mounting supports 306, 308 are curved similar to an outside curvature of the upper and lower frame portions 304 of the frame 200. As such, the ends 310 facilitate alignment of the first and second mounting supports 306, 308 that are coupled or mounted to the upper and lower frame portions 304 of the frame 200. The ends 310 also provide support to maintain the mounting plates 42 in position when secured using the mounting system 300.

The mounting system 300 further includes engagement portions 314 configured to engage with and couple to the mounting plates 42. In the illustrated example, the engagement portions 314 each include openings 316 therethrough configured to receive a fastener (e.g., a bolt 318) to secure to the mounting plates 42 (e.g., the bolt 318 extends through openings in engagement portions 314 and openings in sides of the mounting plates 42). As can be seen, the openings in the mounting plates 42 allows for the bolt 318 to pass therethrough and be secured with a nut to securedly couple the engagement portions 314 to the mounting plates 42. In one or more examples, the engagement portions 314 each have at least one opening configured to receive fasteners to couple to different types of equipment mounts, such as the mounting plates 42. It should be noted that any type of suitable fastener or securing mechanism can be used.

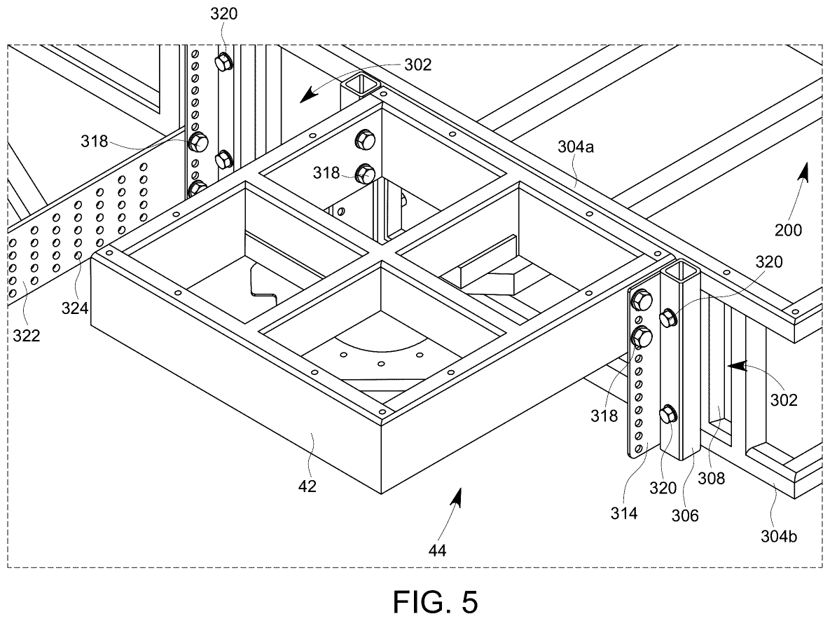

In the illustrated examples, the engagement portion 314 is a plate that extends from the first mounting support 306 of the clamping portion 302. In one example, the engagement portion 314 is coupled or integrated with one side of the first mounting support 306 and extends parallel to the side and perpendicular to the adjacent side of the first mounting support 306. In this configuration, rows of openings 316, illustrated as two rows of openings 316 (configured as mounting holes), extend along a longitudinal axis of the engagement portion 314 to allow for selective positioning of the mounting plates 42 at different heights. That is, FIG. 4 illustrates the mounting plate 42 coupled in a lowermost position of the engagement portion 314 and FIG. 5 illustrates the mounting plate 42 in an uppermost position of the engagement portion 314. As such, the mounting plates 42 can be provided at different mounting heights or locations. The mounting plates 42 can thereby be positioned at different locations along the engagement portion 314, such as at intermediate positions between the positions shown in FIGS. 4 and 5. It should be noted that the number of openings 316, the spacing of the openings 316, the shape of the openings 316, the alignment of the openings 316, etc. can be varied as desired or needed. The engagement portion 314, thus, is configured as a base plate having a plurality of openings 316 arranged in a pair of rows of mounting holes extending longitudinally along the base plate.

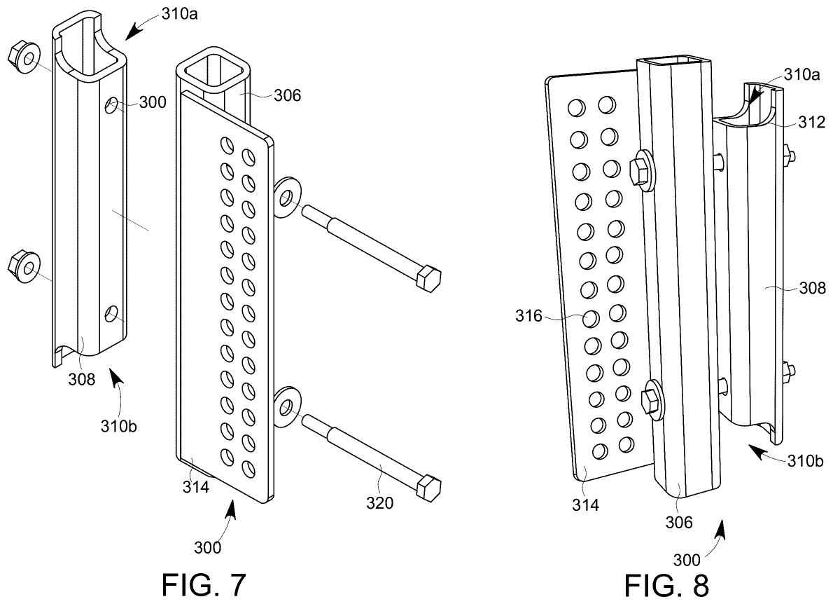

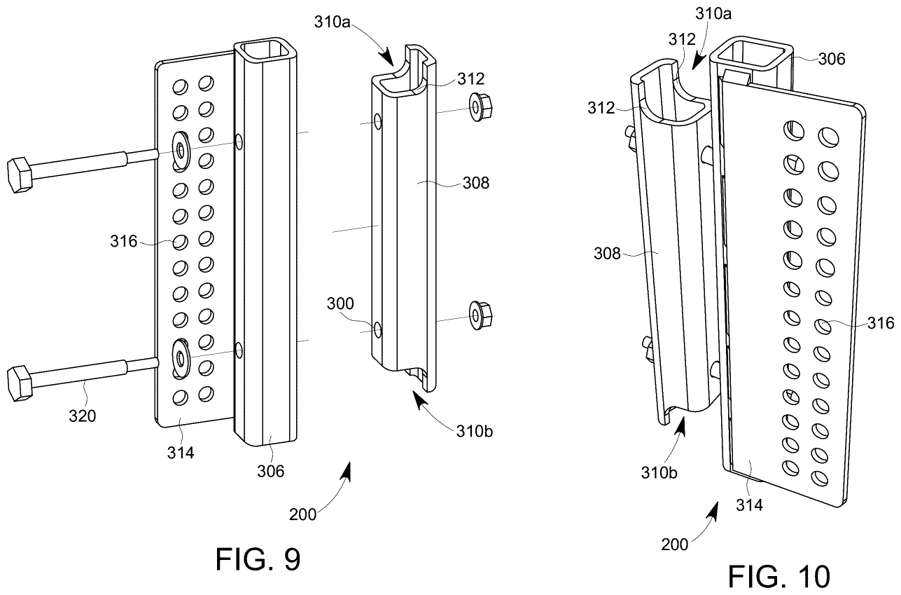

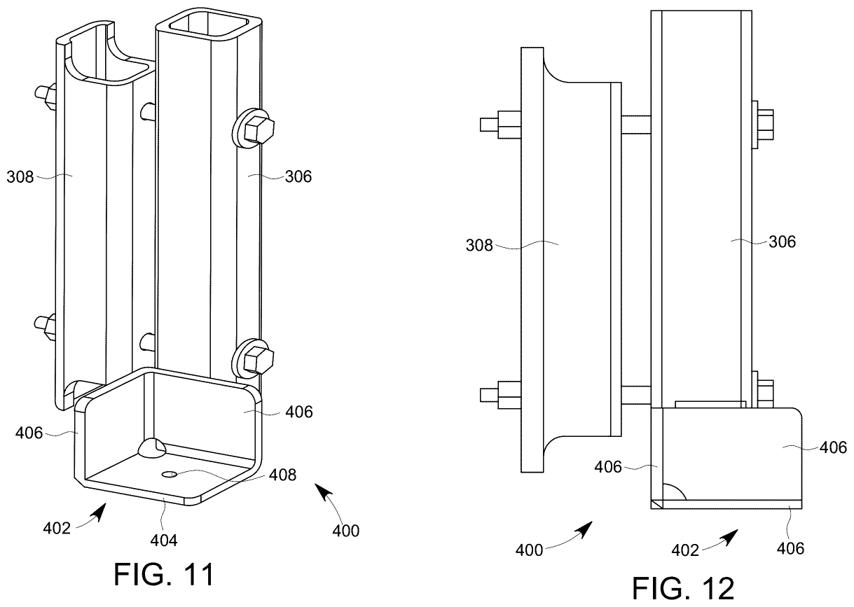

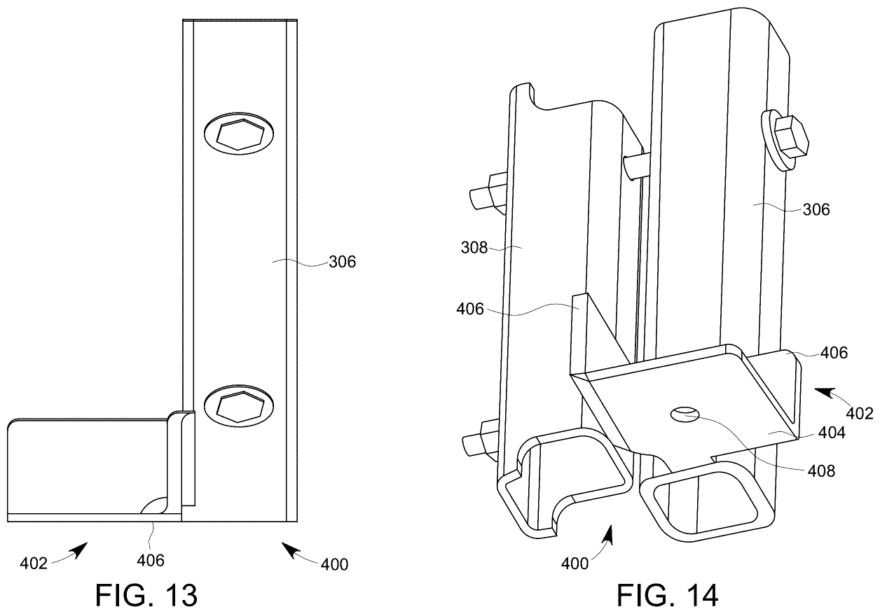

As can be seen more clearly in FIGS. 7-10, the ends 310 of the second mounting support 308 include curved portions 312 configured to engage at least portions of two adjacent sides of the truss structure, wherein the two adjacent sides are in perpendicular planes. For example, the curved portions 312 are sized and shaped in various examples to engage and abut perpendicular sides of the upper and lower frame portions 304 of the frame 200. In the illustrated example, the curved portion 312 at the top end 310a of the second mounting support 308 is configured to engage a side and bottom of the upper frame portion 304a, and the curved portion 312 at the bottom end 310b of the second mounting support 308 is configured to engage a side and a top of the lower frame portion 304b. That is, the ends 310 are configured to engage sides of the upper and lower frame portions 304 of the frame 200 that are in the same plane, and opposing bottom and top surfaces of the upper and lower frame portions 304 of the frame 200. As such, the second mounting support 308 is secured between the upper and lower frame portions 304 of the frame 200 and held against sides of the upper and lower frame portions 304 of the frame 200 when secured to the first mounting support 306 using fasteners, illustrated as bolts 320. For example, the curved portions 312 are in an opposed arrangement and configured to engage opposing top and bottom surfaces of supports of the truss structure, namely of the upper and lower frame portions 304 of the frame 200. It should be noted that different features that are complementary to the frame portion or other portions are also contemplated.

In some examples, the curved portions 312 are L-shaped portions. However, other curved profiles and shapes, or features, can be used that are complementary to the upper and lower frame portions 304 of the frame 200. That is, the curved portions 312 can have different curvatures or multiple curvatures as desired or needed. In some examples, the ends 310 are sized and shaped in a non-curved configuration to engage different sized or shape truss elements or portions.

With reference again in FIGS. 4-6, in some examples, an extension plate 322 is provided and configured to couple with the engagement portion 314. The extension plate 322 allows for mounting of the mounting plates 42, or other equipment support, at a distance from the upper and lower frame portions 304 of the frame 200. For example, in this arrangement, the engagement portion 314 having the openings 316 is configured or operates as a base plate that extends vertically in the mounted position with the extension plate 322 extending horizontally in the mounted position, and extending from the engagement portion 314. As can be seen, similar to the engagement portion 314, the extension plate 322 includes a plurality of openings 324 arranged in rows. In this example, the openings 316 of the engagement portion 314 are arranged in two rows and the openings 324 of the extension plate 322 are arranged in five rows. As can be seen, the extension plate 322 is wider than the engagement portion 314 in this example. In particular, the extension plate 322 in this example has a width that is greater than the width of a side of the mounting plate 42 to which it is coupled. However, the extension plate 322 can have different shapes and sizes as desired or needed, as well as a different number and/or arrangement of openings 324.

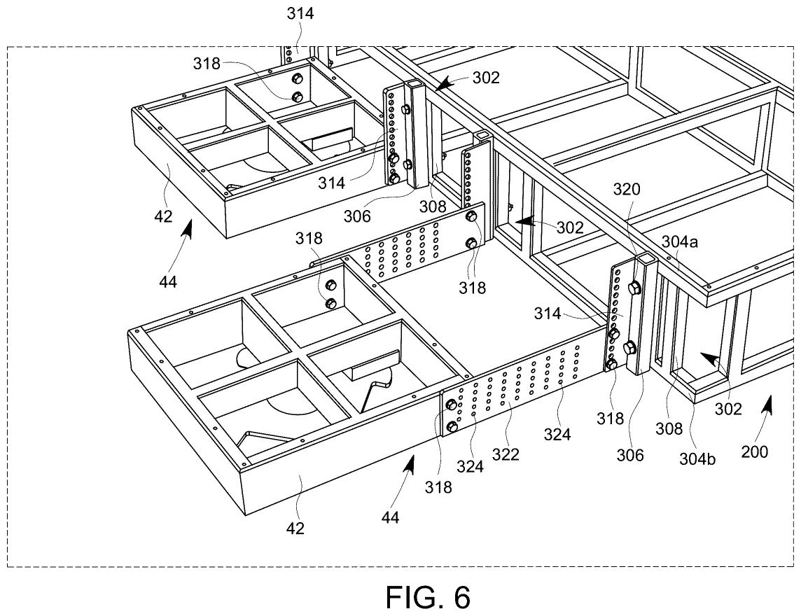

In various examples, the extension plate 322 is coupled to the engagement portion 314 with one or more bolts 318. That is, bolts 318 are used to couple an end of the extension plate 322 to a pair of openings 316 of the engagement portion 314 at a desired or required height. The extension plate 322 is also coupled to the mounting plate 42 with bolts 318 extending through a pair of the openings 324 of the extension plate 322 and through openings in the mounting plate 42. As can be seen in FIG. 4, the mounting plate 42 is coupled at a closest position to the frame 200 and in FIG. 6, the mounting plate 42 is coupled to a farthest most position to the frame 200. As such, the distance between the mounting plate 42 and the frame 200 can be varied as desired or needed. The mounting plates 42 can thereby be positioned at different locations along the extension plate 322, such as at intermediate positions between the positions shown in FIGS. 4 and 6. As such, the vertical and horizontal position of the mounting plate 42 relative to the frame can be varied using one or more of the herein described examples.

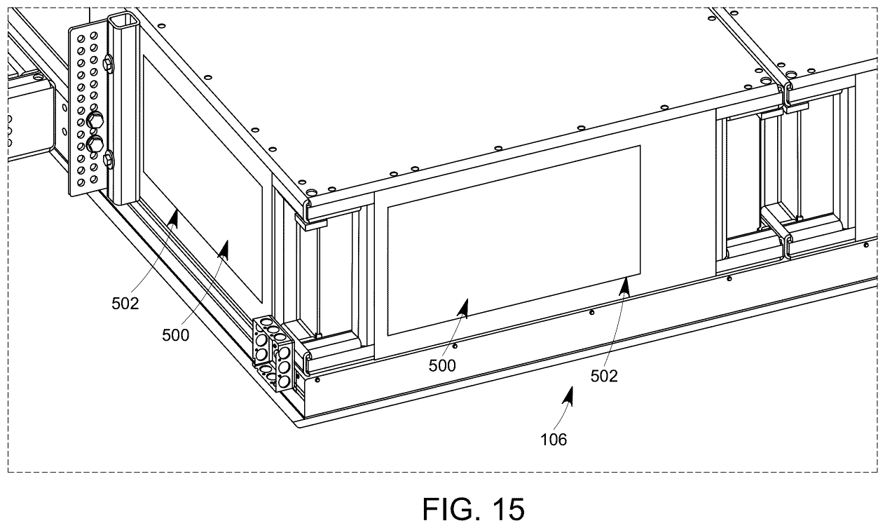

Variations and modification are contemplated, including using different bracket support structures. For example, FIGS. 11-14 illustrate a bracket arrangement 400 configured as a patient lift rail bracket, such as to support a rail of patient lift. In this example, the bracket arrangement 400 includes a corner support member 402 configured to engage and support a rail. That is, the corner support member 402 is sized and shaped in some examples to be complementary to the end of a rail of a patient lift (e.g., features that are complementary) to thereby support one end of the rail. In some examples, the corner support member 402 is configured to support the rail in a parallel relationship or orientation to the frame 200 (e.g., along a side of the frame 200). However, other configurations are contemplated, for example, in an angled or perpendicular relationship or orientation to the frame 200 (e.g., extending away from the frame 200).

In the illustrated example, the corner support member 402 includes a lower wall 404 (e.g., a base wall) and two adjacent side walls 406 perpendicular to the lower wall 404 that together define a corner configuration. That is, the three walls 404, 406 are formed to define a corner bracket or brace for a rail. The corner support member 402 in some examples is a single integrated piece and having one of the side walls 406 coupled to (or formed as part of) the first mounting support 306. In this example, the corner support member 402 is coupled to a bottom end of the first mounting support 306 having the lower wall 404 in a same plane as the bottom end of the first mounting support 306. However, in other examples, the corner support member 402 can be coupled or positioned at different locations along the first mounting support 306.

The lower wall 404 in various examples includes at least one opening 408, illustrated as a single hole. The opening 408 is configured to allow the corner support member 402 to be coupled to a rail or other structure to be supported thereon and maintained in position by the corner configuration (e.g., corner shaped design). The opening 408 in some examples is configured to receive a bolt or other fastener therethrough to secure to the rail or other member. The bracket arrangement 400 having the corner support member 402 is merely another example of a mounting configuration contemplated by the herein described examples. As should be appreciated, other mounting arrangements (e.g., other brackets, supports, etc.) can be implemented in an adjustable manner using the mounting system 300 described herein. That is, the herein described components allow for ease in positioning and coupling of different mounts (e.g., mounting plates 42) to the frame 200 to allow flexibility in design and re-design.

The various components of the mounting system 300 can also be varied as desired or needed. For example, the first mounting support 306 is shown having a first length and the second mounting support 308 is shown having a second length, wherein the first length is greater than the second length. That is, the first mounting support 306 is longer than the second mounting support 308. However, the lengths of the first mounting support 306 and the second mounting support 308 can be varied to have different lengths, including different relative lengths.

The first mounting support 306 is configured to mount to and abut an outer surface of a perimeter of the truss structure (e.g., outer portions of the frame 200) and the second mounting support 308 is configured to mount to and abut an inner surface of the perimeter of the truss structure. It is contemplated that the first mounting support 306 and the second mounting support 308 can be mounted to and/or abut different surfaces of the frame 200 or other structures of the overall system.

The first mounting support 306 and the second mounting support 308 are illustrated as having hollow tubular bodies with a square shaped profile. However, the first mounting support 306 and the second mounting support 308 can have different shapes and configurations, and be partially hollow or solid. For example, the first mounting support 306 and the second mounting support 308 can have different cross-sectional shapes or profiles, such as based on the structure or configuration of the frame 200. In various examples, the first mounting support 306 and the second mounting support 308 are configured to allow mounting to any location along a perimeter of the truss structure, for example, at different locations along the frame 200.

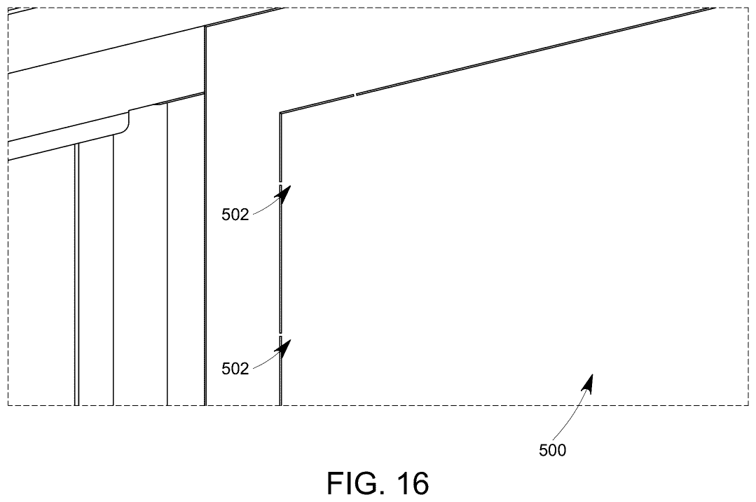

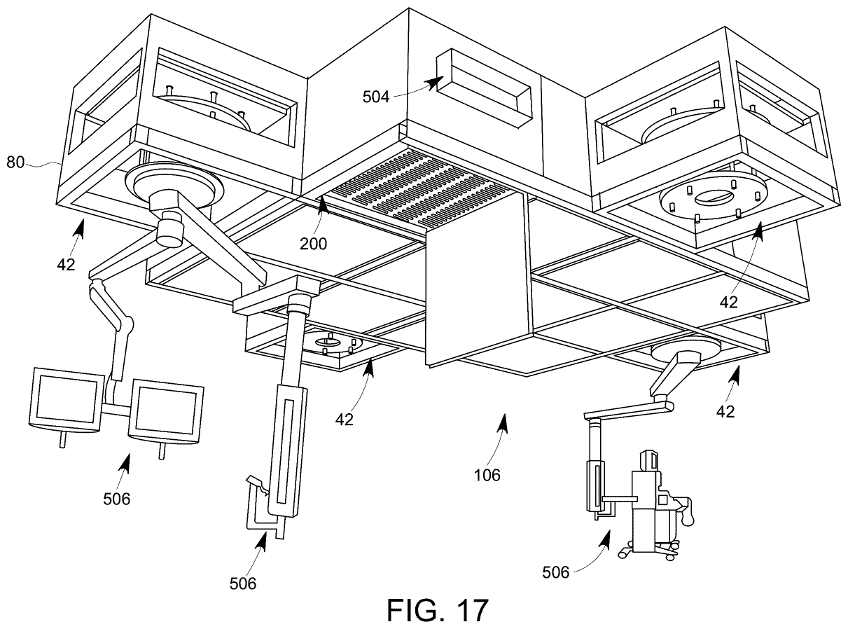

In one or more examples, additional or optional flexibility in design or configuration of the supply air array 106 (that includes the frame 200 in some examples) is provided, such as shown in FIGS. 15 and 16. In various examples, the supply air array 106 forms an airframe system with the frame 200 or other support structure and includes a plurality of removable portions 500, which are configured as “knock-outs” or other portions that are capable of being removed to form or expose openings into the supply air array 106 (e.g., airflow inlets). For example, the supply air array 106 includes the plurality of removable portions 500 positioned at airflow inlets of the supply air array and covering the airflow inlets. The removable portions 500 can be positioned along one or more sides of the supply air array 106 and/or along the top or bottom of the supply air array 106.

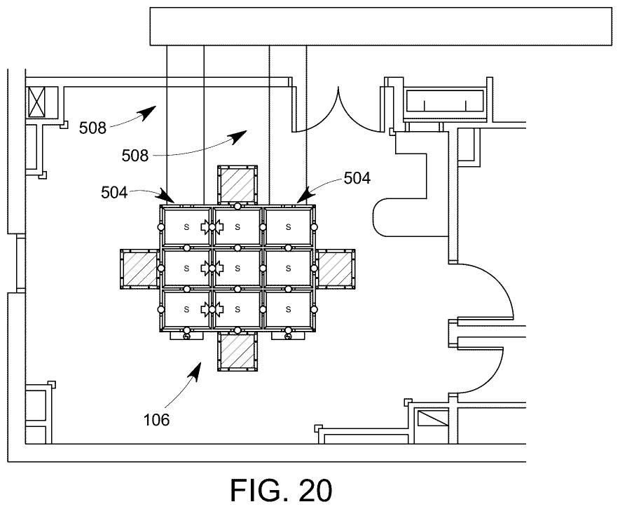

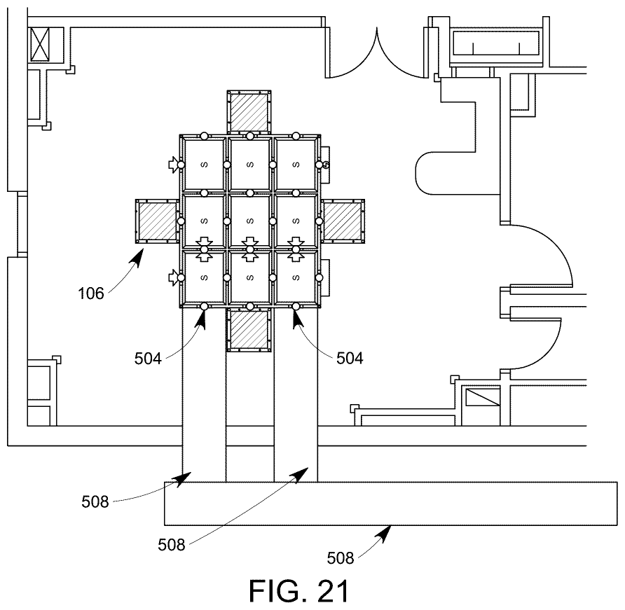

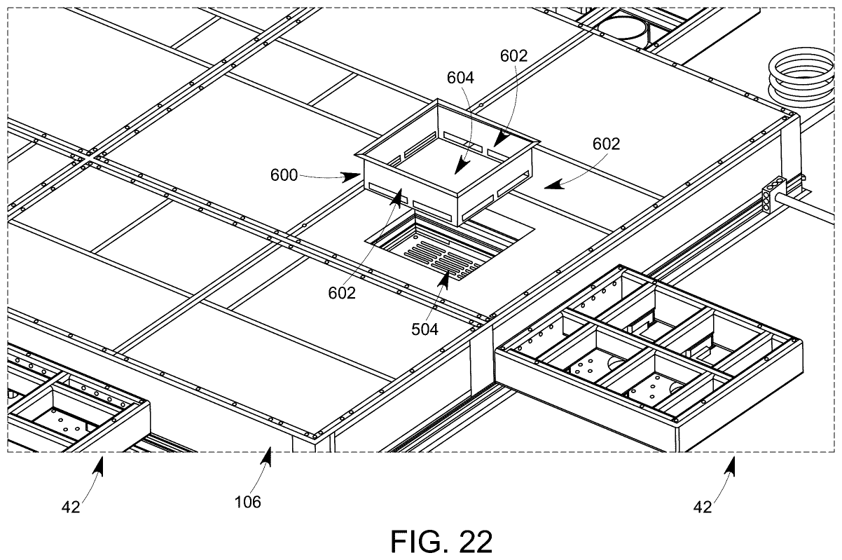

The removable portions 500 in some examples are formed as a plurality of removable supply air panels than can be knocked-out or otherwise removed to provide airflow communication or an airflow path into the interior of the supply air array 106. In some examples, the supply air array 106 defines a plenum for supply of air, such as described in U.S. Pat. Nos. 9,903,115, 10,405,952, and 11,259,893. As can be seen, the removable portions 500 in various examples are supply air panels positioned in a spaced apart configuration along one or more sides of, for example, the frame 200, and in a spaced apart configuration along a top of the frame 200 as illustrated in FIG. 19. The removable portions 500 are formed within the surfaces of the supply air array 106 by perforations 502 (e.g., microtabs) that define a shape and size of the removable portions 500. That is, the removable portions 500 are configured to have a shape that defines an airflow inlet in some examples and that allows for coupling to an incoming air ductwork 508 as illustrated in FIGS. 20 and 21.

For example, as can be seen in the configuration of the supply air array 106 shown in FIG. 20, two removable portions 500 have been removed to define airflow inlets 504 to connect to the air ductwork 508 at a top of the room as viewed in this figure. As can be seen in the configuration of the supply air array 106 shown in FIG. 21, two removable portions 500 have been removed to define airflow inlets 504 to connect to the air ductwork 508 at a bottom of the room as viewed in this figure. That is, different airflow inlets 504 have been configured based on the arrangement of the air ductwork 508 in the room to allow connection and airflow communication between the supply air array 106 and the air ductwork 508. Accordingly, in various examples, air supply panels, namely removable portions 500 are pulled or taken out (removed) based on the direction and/or location of the incoming supply air ductwork 508 to allow airflow to be configurably supplied to the supply air array 106. As should be appreciated, airflow inlets 504 can be configured at the various different locations defined by the removable portions 500, which in some examples, are provided along one or more sides (or all sides), the top and/or the bottom of the supply air array 106. In some examples, the removable portions 500 are configured or positioned in typical industry locations for supply air inlets. In other examples, the removable portions 500 are optionally or alternatively positioned in other locations. It should be appreciated that only the removable portions 500 needed for airflow connection are removed in various examples.

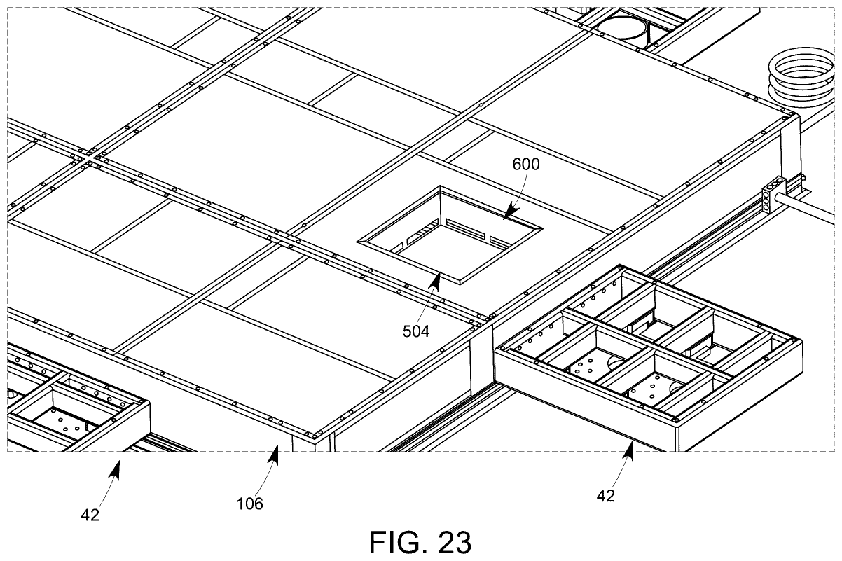

It should be noted that in configurations wherein the airflow inlet 504 is provided on the top of the supply air array 106 by removing a panel (removable portion 500) on the top of the supply air array 106, an air control device is provided as illustrated in FIGS. 22 and 23. In the illustrated example, a blast plate cartridge 600 is provided when a top supply air entry configuration is used. That is, the blast plate cartridge 600 is configured to be inserted within the opening formed by the airflow inlet 504 (e.g., top airflow inlet) to control the airflow into the supply air array 106. In some examples, the blast plate cartridge 600 includes a plurality of slots 602 or other openings along a bottom portion of the sides of the blast plate cartridge 600 and having a solid base 604. As can be seen, the blast plate cartridge 600 includes two slots 602 along each side of the slots 602 near the bottom of the blast plate cartridge 600. However, additional or fewer slots 602 can be provided.

In this configuration, the airflow, such as from the air ductwork 508, is controlled to prevent air blast into the supply air array 106. For example, the configuration of the blast plate cartridge 600 provides even airflow into the supply air array 106 by controlling airflow to be directed along the entire face of the base 604. That is, the blast plate cartridge 600 creates airflow resistance and pressure, and redirects air to evenly redistribute air within the blast plate cartridge 600 (e.g., air velocity is controlled down a shallow field and a hard redirection is provided by the solid base 604 and through the slots 602). As such, the blast plate cartridge 600 provides an air control module in some examples that is inserted within the airflow inlet 504 when an overhead air supply is used. It should be noted that the blast plate cartridge 600 is configured to be complementary to the size and shape of the airflow inlet 504.

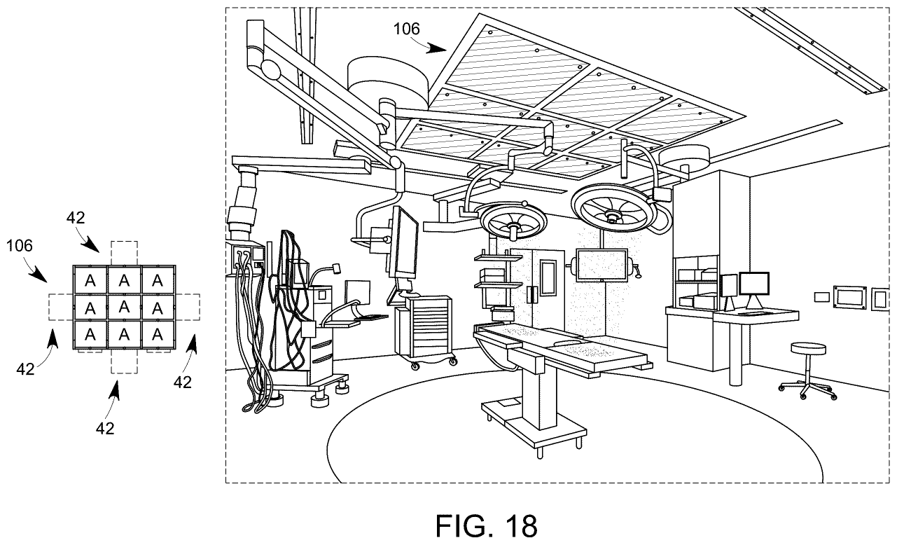

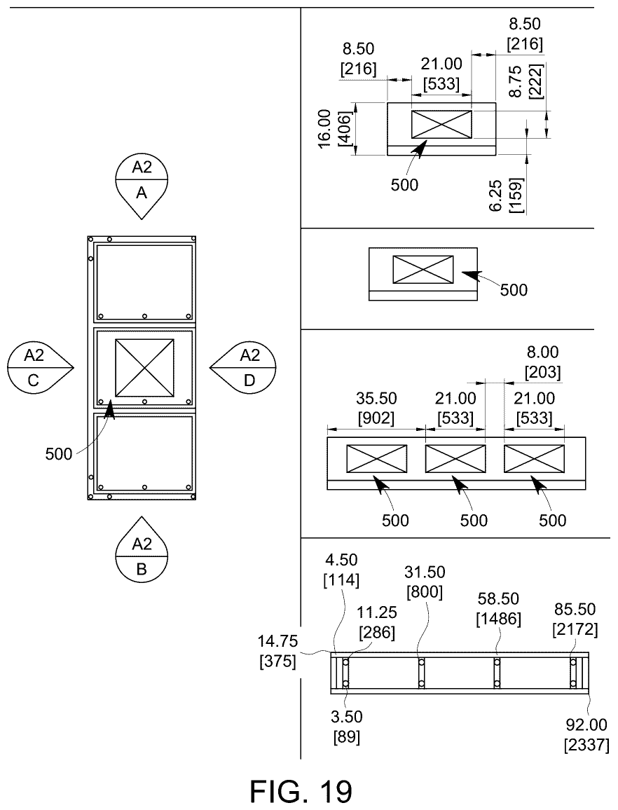

Thus, as shown in FIG. 17, the supply air array 106 has the airflow inlet 504 formed by removing one of the removable portions 500. As can be seen, the supply air array 106 is formed as part of the frame 200 and includes equipment 506 supported by mounting plates 42 coupled to the frame 200 as described in more detail herein. As should be appreciated, the position of the my mounting plates 42 can be configured on the fly or adjusted on the fly using the mounting arrangement described in more detail herein. Additionally, the location of the airflow inlet 504 likewise can be configured on the fly or adjusted on the fly using the removable portions 500 as described in more detail herein (e.g., the plurality of removable supply air panels are coupled with the frame 200 using the plurality of perforations 502 (e.g., microtabs) to allow easy knockout of the panels). FIG. 18 illustrates a configuration with four mounting plates 42 coupled to the frame 200 within a medical room.

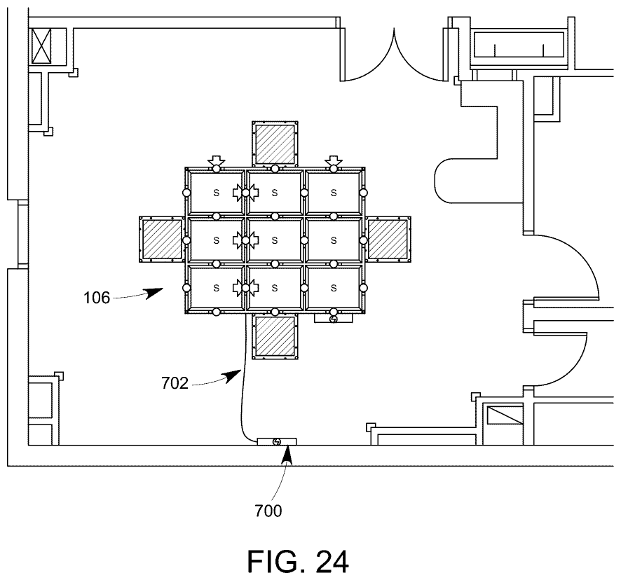

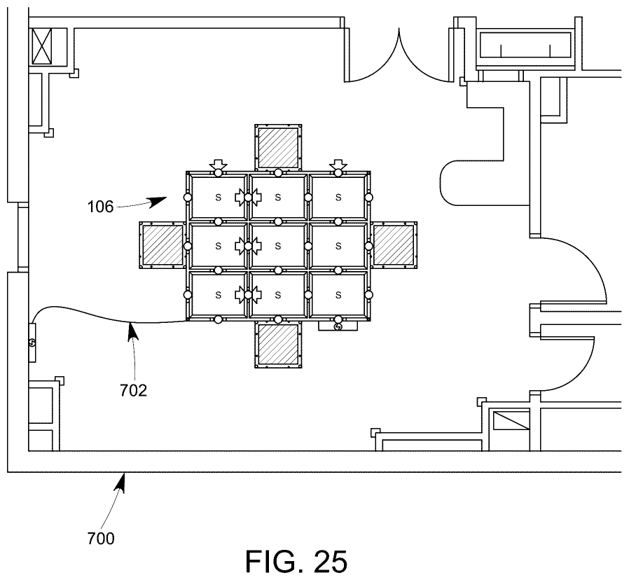

In some examples, other configurable aspects are provided. For example, as shown in FIGS. 24 and 25, a movable electrical enclosure 700 with a flexible electrical connection member 702 is provided. As can be seen, with the adjustable mounting arrangement described herein, the movable electrical enclosure 700 can be coupled at different locations of the supply air array 106 using the flexible electrical connection member 702. In some examples, the flexible electrical connection member 702 is a power whip of other electrical cable (e.g., a ten foot power whip). The electrical enclosure in some examples can be moved to a remote location if desired or required. Thus, electrical connection configurability is also provided in various examples.

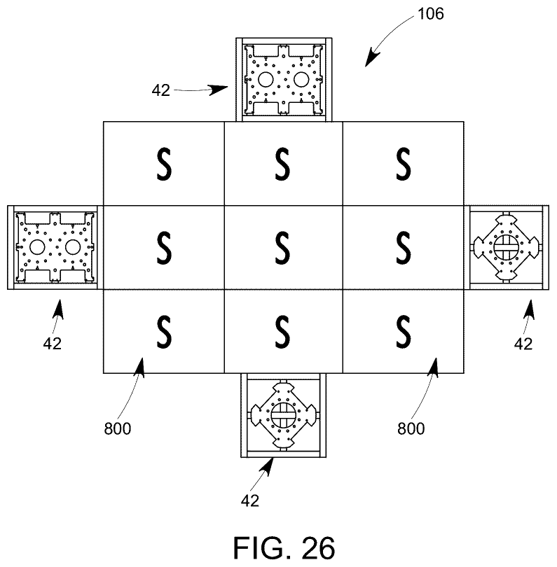

In one or more examples, the configuration of the supply air array 106 is pre-defined and can be selected from a plurality of different pre-defined arrangements. For example, the frame 200 includes a plurality of elements defining the supply air array 106, wherein the plurality of elements are arranged in defined or pre-defined configuration that have airflow paths determined. That is, in some examples, the airflow math and/or models are precomputed to determine the aspect ratio that operates optimally for the particular room configuration. The pre-defined configurations have the plurality of elements arranged based on a model type in some examples, such as having the plurality of elements being a plurality of modules 800 arranged in a pre-defined shape or design as illustrated in FIG. 26. As such, in some examples, a product catalog with predefined base options for the supply air array 106 can be selected with the configurability of the various equipment mounts provided as described in more detail herein. For example, standard catalog models of the supply air array 106 are preconfigured and engineered to meet current guidelines and the airflow math is precomputed for each of the various configurations. As should be appreciated, the preconfigured supply air arrays 106 in some exampled define models that are sized to meet a variety of room dimensions, while providing flexibility in equipment mounting as described herein. In some examples, all of the preconfigured supply air arrays 106 are configured to use one standard HEPA air filter.

Thus, various examples provide the supply air array 106 with a flexible or configurable mounting system that allows, for example, for easily configuring and changing the location of frame or ceiling mounted components. The mounting system allows for adjusting the mounting locations along the frame structure or truss. In some examples, pre-defined configurations of modules are provided that allow for additional configurability as described herein. The equipment mounts are adjustably located along the perimeter of the system in various examples, thereby being located along the perimeter of the airfield. In this configuration, air flow within the airfield is improved by not having the mounts within the portion of the system that includes the airfield.

Examples described herein may be used in relation to a hospital operating room environment or other medical setting. Optionally or alternatively, examples described herein may be used in various other settings in which pressurized airflow may be directed in combination with ceiling mounted equipment and/or lighting assemblies. For example, one or more examples may be used in dental offices, manufacturing clean rooms, residential spaces, and the like. Additionally, it should also be appreciated that one or more air filtering, air sterilizing and/or air purifying devices or methods may be used in combination with each other and in the various examples.

Based on the foregoing, the following provides a general overview of the present disclosure and is not a comprehensive summary:

Clause 1. A mounting system comprising: a clamping portion configured to removably couple to a truss structure, the clamping portion having a first mounting support and a second mounting support, the first and second mounting supports configured to couple to each other on opposite sides of a portion of the truss structure, and the second mounting support having features complimentary to a shape of the truss structure to allow sliding engagement of the truss structure; and an engagement portion having at least one opening, wherein the at least one opening is configured to receive one or more fasteners to couple to an equipment mount, and the engagement portion extends from the first mounting support of the clamping portion.

Clause 2. The mounting system of clause 1, wherein the engagement portion comprises a base plate having a plurality of openings arranged in a pair of rows of mounting holes extending longitudinally along the base plate.

Clause 3. The mounting system of one or more of clauses 1 and 2, wherein the complimentary features are located at ends of the second mounting support and comprise curved portions configured to engage two adjacent sides of the truss structure, wherein the two adjacent sides are in perpendicular planes.

Clause 4. The mounting system of one or more of clauses 1-3, wherein the curved portions are L-shaped portions.

Clause 5. The mounting system of one or more of clauses 1-4, wherein the curved portions are in an opposed arrangement and configured to engage opposing top and bottom surfaces of supports of the truss structure.

Clause 6. The mounting system of one or more of clauses 1-5, wherein the engagement portion comprises a base plate having the plurality of openings, the base plate extending vertically in a mounted position, and further comprising an extension plate having a plurality of openings, the extension plate extending from and configured to couple to the base plate and extend horizontally in the mounted position.

Clause 7. The mounting system of one or more of clauses 1-6, wherein the engagement portion comprises a corner support member having a lower wall and two adjacent side walls perpendicular to the lower wall, wherein the at least one opening is located in the lower wall, the corner support member extending from the first mounting support of the clamping portion.

Clause 8. The mounting system of one or more of clauses 1-7, wherein the first mounting support has a first length and the second mounting portion has a second length, wherein the first length is greater than the second length.

Clause 9. The mounting system of one or more of clauses 1-8, wherein the first mounting support is configured to mount to and abut an outer surface of a perimeter of the truss structure and the second mounting support is configured to mount to and abut an inner surface of the perimeter of the truss structure.

Clause 10. The mounting system of one or more of clauses 1-9, wherein the first and second mounting supports have hollow tubular bodies with a square shaped profile.

Clause 11. The mounting system of one or more of clauses 1-10, wherein the first and second mounting supports are configured to mount to any location along a perimeter of the truss structure.

Clause 12. An airframe system comprising: a frame defining a supply air array having a plurality of removable portions, wherein the plurality of removable portions are positioned at airflow inlets of the supply air array and covering the airflow inlets; and one or more equipment mounts movably mounted to a perimeter of the frame.

Clause 13. The airframe system of clause 12, wherein the plurality of removable portions comprise a plurality of supply air panels.

Clause 14. The airframe system of one or more of clauses 12 and 13, wherein the plurality of supply air panels are positioned in a spaced apart configuration along one or more sides of the frame, and in a spaced apart configuration along a top of the frame.

Clause 15. The airframe system of one or more of clauses 12-14, wherein the airflow inlets are configured to couple to an incoming air ductwork.

Clause 16. The airframe system of one or more of clauses 12-15, wherein the plurality of supply air panels are formed in the supply air array with a plurality of microtabs.

Clause 17. The airframe system of one or more of clauses 12-16, further comprising a movable electrical enclosure with a flexible electrical connection member.

Clause 18. The airframe system of one or more of clauses 12-17, further comprising the mounting system of claim 1.

Clause 19. The airframe system of one or more of clauses 12-18, wherein the frame comprises a plurality of elements defining the supply air array, the plurality of elements arranged in pre-defined configuration.

Clause 20. The airframe system of one or more of clauses 12-19, wherein the pre-defined configuration has the plurality of elements arranged based on a model type.

Clause 21. The airframe system of one or more of clauses 12-20, wherein the plurality of elements comprise a plurality of modules arranged in a pre-defined shape.

While various spatial and directional terms, including but not limited to top, bottom, lower, mid, lateral, horizontal, vertical, front and the like are used to describe the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

Any range or value given herein can be extended or altered without losing the effect sought, as will be apparent to the skilled person.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A mounting system comprising:
a clamping portion configured to removably couple to a truss structure, the clamping portion having a first mounting support and a second mounting support, the first and second mounting supports configured to couple to each other on opposite sides of a portion of the truss structure, and the second mounting support having features complimentary to a shape of the truss structure to allow sliding engagement of the truss structure; and
an engagement portion having at least one opening, wherein the at least one opening is configured to receive one or more fasteners to couple to an equipment mount, and the engagement portion extends from the first mounting support of the clamping portion.

2. The mounting system of claim 1, wherein the engagement portion comprises a base plate having a plurality of openings arranged in a pair of rows of mounting holes extending longitudinally along the base plate.

3. The mounting system of claim 1, wherein the complimentary features are located at ends of the second mounting support and comprise curved portions configured to engage two adjacent sides of the truss structure, wherein the two adjacent sides are in perpendicular planes.

4. The mounting system of claim 3, wherein the curved portions are L-shaped portions.

5. The mounting system of claim 3, wherein the curved portions are in an opposed arrangement and configured to engage opposing top and bottom surfaces of supports of the truss structure.

6. The mounting system of claim 2, wherein the engagement portion comprises a base plate having the plurality of openings, the base plate extending vertically in a mounted position, and further comprising an extension plate having a plurality of openings, the extension plate extending from and configured to couple to the base plate and extend horizontally in the mounted position.

7. The mounting system of claim 1, wherein the engagement portion comprises a corner support member having a lower wall and two adjacent side walls perpendicular to the lower wall, wherein the at least one opening is located in the lower wall, the corner support member extending from the first mounting support of the clamping portion.

8. The mounting system of claim 1, wherein the first mounting support has a first length and the second mounting portion has a second length, wherein the first length is greater than the second length.

9. The mounting system of claim 1, wherein the first mounting support is configured to mount to and abut an outer surface of a perimeter of the truss structure and the second mounting support is configured to mount to and abut an inner surface of the perimeter of the truss structure.

10. The mounting system of claim 1, wherein the first and second mounting supports have hollow tubular bodies with a square shaped profile.

11. The mounting system of claim 1, wherein the first and second mounting supports are configured to mount to any location along a perimeter of the truss structure.

12. An airframe system comprising:
a frame defining a supply air array having a plurality of removable portions, wherein the plurality of removable portions are positioned at airflow inlets of the supply air array and covering the airflow inlets; and
one or more equipment mounts movably mounted to a perimeter of the frame.

13. The airframe system of claim 12, wherein the plurality of removable portions comprise a plurality of supply air panels.

14. The airframe system of claim 13, wherein the plurality of supply air panels are positioned in a spaced apart configuration along one or more sides of the frame, and in a spaced apart configuration along a top of the frame.

15. The airframe system of claim 13, wherein the airflow inlets are configured to couple to an incoming air ductwork.

16. The airframe system of claim 13, wherein the plurality of supply air panels are formed in the supply air array with a plurality of microtabs.

17. The airframe system of claim 12, further comprising a movable electrical enclosure with a flexible electrical connection member.

18. The airframe system of claim 12, further comprising the mounting system of claim 1.

19. The airframe system of claim 12, wherein the frame comprises a plurality of elements defining the supply air array, the plurality of elements arranged in pre-defined configuration.

20. The airframe system of claim 19, wherein the pre-defined configuration has the plurality of elements arranged based on a model type.

21. The airframe system of claim 19, wherein the plurality of elements comprise a plurality of modules arranged in a pre-defined shape.
